(19) United States
(12) Patent Application Publication
(10) Pub. No.: US 2013/0137133 A1
(43) Pub. Date: May 30, 2013

(54) ULTRASONIC DISSECTION DEVICE AND ULTRASONIC DISSECTION METHOD

(71) Applicant: OLYMPUS CORPORATION, Tokyo (JP)

(72) Inventors: Yasuo SASAKI, Machida-shi (JP); Yoshihiko WATANABE, Yokohama-shi (JP)

(73) Assignee: OLYMPUS CORPORATION, Tokyo (JP)

(21) Appl. No.: 13/750,028

(22) Filed: Jan. 25, 2013

Related U.S. Application Data
(62) Division of application No. 12/503,331, filed on Jul. 15, 2009.

(30) Foreign Application Priority Data
Jul. 16, 2008 (JP) 2008-184738

(51) Int. Cl. C12Q 1/24

(52) U.S. Cl. CPC C12Q 1/24
USPC 435/30

(57) ABSTRACT

There is provided an ultrasonic dissection device for relatively removing a target biological object from other biological objects, the ultrasonic dissection device including an ultrasonic wave generation unit, an ultrasonic wave convergence unit, and a controller to control the ultrasonic wave generation unit.

### CROSS-REFERENCE TO RELATED APPLICATIONS

This application is a divisional application of U.S. patent application Ser. No. 12/503,331 filed on Jul. 15, 2009 which is based upon and claims the benefit of priority from prior Japanese Patent Application No. 2008-184738, filed Jul. 16, 2008, the entire contents of each of which are incorporated herein by reference.

### BACKGROUND OF THE INVENTION

1. Field of the Invention

The present invention relates to an ultrasonic dissection device and an ultrasonic dissection method for generating acoustic streaming by a converged ultrasonic wave in a solution containing a mass of biological objects arranged on a flat carrier, thereby removing a target biological object from the mass of biological objects.

2. Description of the Related Art

Recently, a study of harvesting single or a few cells or minute living tissue to analyze genes therein has become active. For example, a study of harvesting a cell in tumor tissue by microdissection to analyze a gene expression of only a tumor cell and to analyze a gene expression level in adjacent individual nerve cells has been done. Especially, it becomes important to analyze at a single cell level in a cancer cell, a nerve cell, an embryo cell and a stem cell, and a technique to harvest a living specimen such as the single cell and the minute living tissue is an important technique to dominate accuracy of the analysis thereafter.

As a dissection technique to cut out a required cell from a cell group, cutting by a UV laser (Jpn. Pat. Appln. KOKAI Publication No. 2002-156316 and U.S. Pat. No. 5,998,129), a method of bonding to a surface of an adhesive allowed to contact the cell by heat by an IR laser (Japanese Patent No. 3786711), and a method of cutting the cell with a vibrated thin bar (Jpn. Pat. Appln. KOKAI Publication No. 2004-305441) are known so far. However, the method with the UV laser has a phototoxic problem to a living cell, and the method with the IR laser has a heat problem, so that it is difficult to apply them to the living cell. Also, although the method with the vibrated thin bar is applicable to the living cell, this method lacks accuracy for correctly taking out the required cell. In addition, the method requires operation with a manipulator, so that an advanced procedure is required.

Also, it is known that the acoustic streaming may be generated under water by the converged ultrasonic wave (The Technical Report UE93-93, EA93-93 (1994-01), The Institute of Electronics, Information and Communication Engineers); however, it is not known that this is applicable to the cell dissection.

### BRIEF SUMMARY OF THE INVENTION

An object of the invention is to provide a dissection device and a dissection method with low invasiveness to a biological specimen such as a cell.

The inventor of the present invention has found that the acoustic streaming generated in liquid by the converged ultrasonic wave may cause the dissection of the cell and completed the present invention.

That is, according to an aspect, the present invention provides an ultrasonic dissection device for generating acoustic streaming by a converged ultrasonic wave in a solution containing a mass of biological objects spread on a flat carrier, thereby relatively removing a target biological object from other biological objects, the ultrasonic dissection device comprising:

ultrasonic wave generation means;

ultrasonic wave convergence means for converging an ultrasonic wave generated by the ultrasonic wave generation means on a boundary of the target biological object and other biological objects; and

a controller to control the ultrasonic wave generation means such that the ultrasonic wave converged by the ultrasonic wave convergence means generates the acoustic streaming having a converged spot diameter effective for relatively removing the target biological object.

According to another aspect, the invention provides an ultrasonic dissection method for generating acoustic streaming by a converged ultrasonic wave in a solution containing a mass of biological objects spread on a flat carrier, thereby relatively removing a target biological object from other biological objects, the method including:

a step of converging an ultrasonic wave on a boundary of the target biological object and other biological objects; and

a step of generating the acoustic streaming having a converged spot diameter effective for relatively removing the target biological object, in the solution containing the mass of biological objects by the converged ultrasonic wave to relatively remove the target biological object from other biological objects.

According to the present invention, novel dissection device and method with low invasiveness to the cell are provided. The device of the present invention is capable of correctly cutting out the cell without requiring the advanced procedure, and is excellent in that the target cell may be dissected at a single cell level.

Advantages of the invention will be set forth in the description which follows, and in part will be obvious from the description, or may be learned by practice of the invention. Advantages of the invention may be realized and obtained by means of the instrumentalities and combinations particularly pointed out hereinafter.

### BRIEF DESCRIPTION OF THE SEVERAL VIEWS OF THE DRAWING

The accompanying drawings, which are incorporated in and constitute a part of the specification, illustrate embodiments of the invention, and together with the general description given above and the detailed description of the embodiments given below, serve to explain the principles of the invention.

FIG. 1 is a view showing an ultrasonic dissection device according to a first embodiment of the present invention;

FIG. 2 is a view showing one example of ultrasonic wave generation means and ultrasonic wave convergence means;

FIG. 3 is a view showing the ultrasonic dissection device provided with illumination means arranged to directly illuminate biological objects;

FIG. 4 is a view showing the ultrasonic dissection device provided with the illumination means arranged to illuminate an ultrasonic wave exit end face of an acoustic lens;

FIG. 5 is a view showing the ultrasonic dissection device used in an example;

FIG. 6 is a timing chart of an electrical signal generated by a signal generator, a signal of an arbitrary signal generator, and a signal thereby output from an RF switch;

FIG. 7 shows photographs showing a result of ultrasonic dissection;

FIG. 8 shows photographs showing the result of the ultrasonic dissection; and

FIG. 9 is a graph showing an experimental result regarding a duration of the ultrasonic wave.

### DETAILED DESCRIPTION OF THE INVENTION

In the present invention, the term “dissection” is intended to mean removal of a target biological object from a mass of biological objects spread on a flat carrier, and one or a plurality of biological objects may be herein removed. In the present invention, the “biological object” is an arbitrary biological specimen spread on the flat carrier, such as an arbitrary cell spread on the flat carrier to form a monolayer, which may be obtained by allowing the cell to grow on a flat surface on the flat carrier. Alternatively, the “biological object” may be a tissue section removed from living tissue and put on the flat carrier. The “carrier” is the flat carrier for holding a solution containing the biological objects, and may be, for example, a tabular substrate for holding the solution containing the biological objects (such as AmpliGrid [manufactured by Advalytix]) or an arbitrary container of which bottom surface is flat and formed of a transparent surface, which transmits visible light, such as a petri dish.

1. Ultrasonic Dissection Device

### First Embodiment

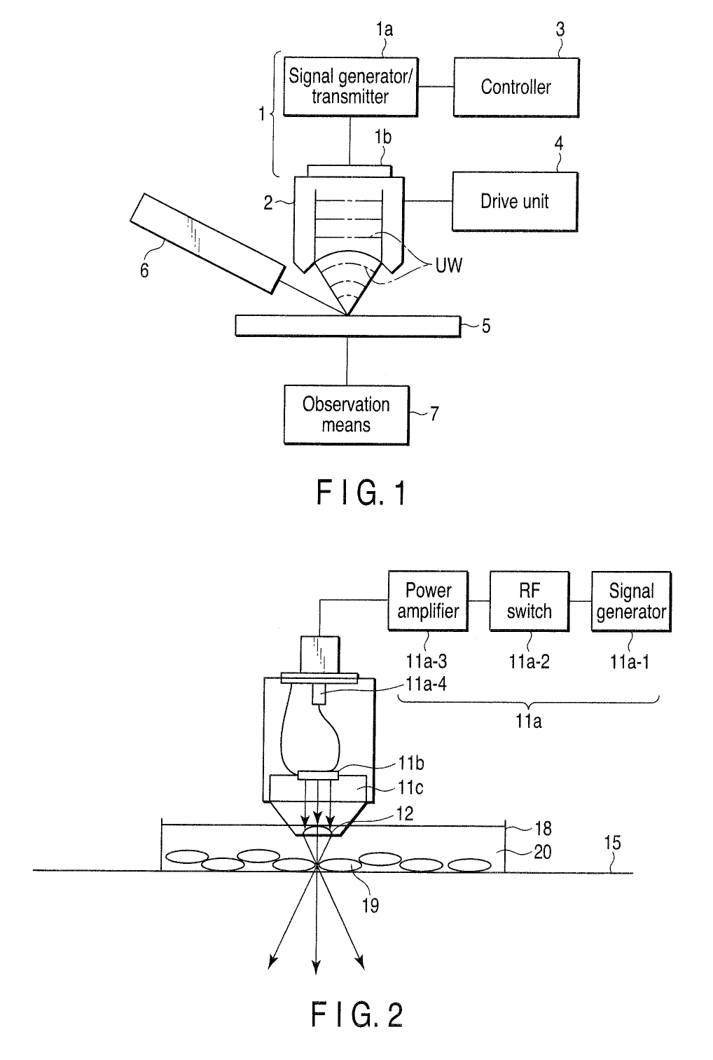

Hereinafter, an ultrasonic dissection device according to a first embodiment of the present invention will be described with reference to FIG. 1.

Meanwhile, the following description is for describing the present invention and is not for limiting the present invention.

As shown in FIG. 1, the ultrasonic dissection device of this embodiment is provided with the following components:

ultrasonic wave generation means 1;

ultrasonic wave convergence means 2 for converging an ultrasonic wave generated by the ultrasonic wave generation means 1;

a controller 3 to control the ultrasonic wave generation means such that the ultrasonic wave converged by the ultrasonic wave convergence means 2 generates acoustic streaming;

a drive unit 4 to drive the ultrasonic wave convergence means 2;

a specimen stage 5 on which a carrier for holding a solution containing biological objects is placed;

illumination means 6 for illuminating the biological objects; and

observation means 7 for observing the biological objects.

Hereinafter, each component and operation of the device according to the first embodiment will be described in the order of the components described above.

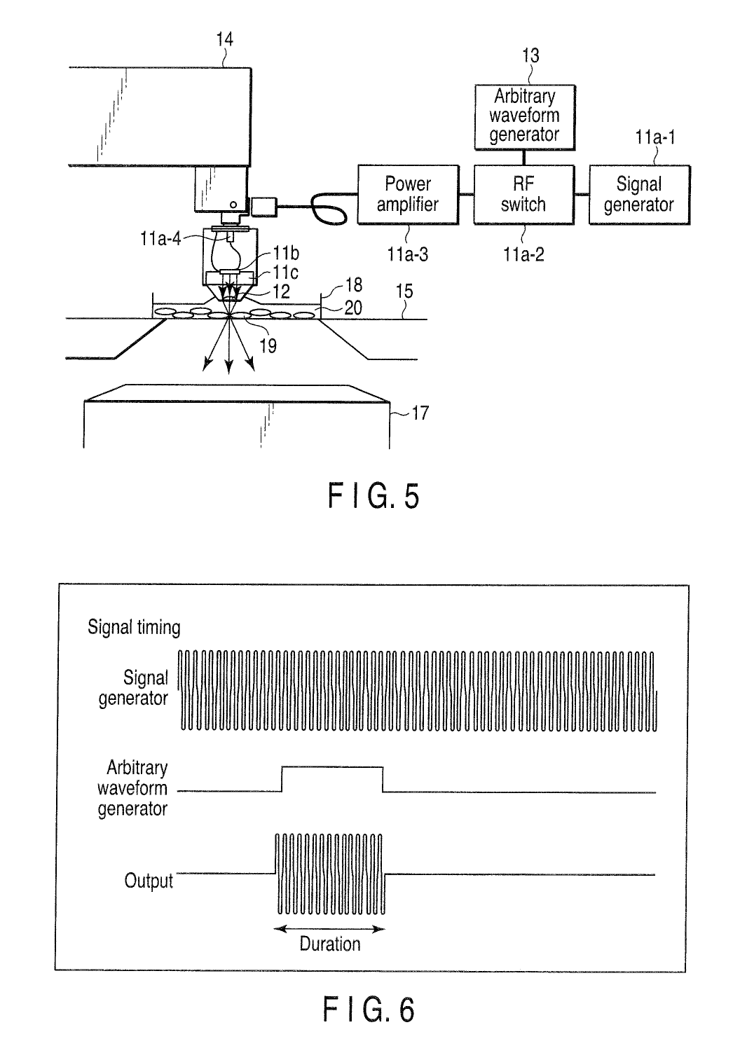

The ultrasonic wave generation means 1 generates an ultrasonic wave. In FIG. 1, the ultrasonic wave generation means 1 is composed of a signal generator/transmitter 1a which generates/transmits a high-frequency signal (electrical signal) having a predetermined frequency, and a transducer (electrical/acoustic transducing element) 1b which transduces the electrical signal to an acoustic wave. The signal generator/transmitter 1a is composed of a signal generator 11a-1 which generates an electrical signal, an RF switch 11a-2 which controls length of the electrical signal based on an output waveform from an arbitrary waveform generator 13, a power amplifier 11a-3 which amplifies an output (power), and a connector 11a-4 which connects the power amplifier 11a-3 and the transducer 1b in a subsequent stage, as shown in FIG. 5. The transducer 1b propagates the acoustic wave corresponding to the high-frequency signal (electrical signal) to the ultrasonic wave convergence means 2 through an ultrasonic wave propagation medium (such as sapphire). ZnO and LiNbO₃ are sputtered on or bonded to the transducer 1b.

The ultrasonic wave convergence means 2 converges the ultrasonic wave generated by the ultrasonic wave generation means 1. The ultrasonic wave convergence means 2 is an acoustic lens, and an ultrasonic wave exit end face thereof forms four spherical surfaces. The ultrasonic wave convergence means 2 deflects an ultrasonic wave (UW) from the transducer 1b such that this converges on a predetermined focal position (boundary of a target biological object and other biological objects) and emits the same. Meanwhile, as used herein, the term boundary includes not only the boundary of the target biological object and other biological objects but also neighborhood of the boundary (mainly, outside of the target biological object).

FIG. 2 shows one example of the ultrasonic wave generation means and the ultrasonic wave convergence means. In FIG. 2, the ultrasonic wave generation means is composed of a signal generator/transmitter 11a which generates/transmits an electrical signal, a ZnO thin-film transducer 11b which transduces the generated electrical signal to an acoustic wave, and an ultrasonic wave propagation medium (sapphire rod) 11c which propagates the acoustic wave to an acoustic lens. The signal generator/transmitter 11a is composed of the signal generator 11a-1, the RF switch 11a-2, the power amplifier 11a-3, and the connector 11a-4. The transducer 11b is preferably formed on a surface opposite to the acoustic lens to have a size similar to that of the acoustic lens. In FIG. 2, the ultrasonic wave convergence means is an acoustic lens 12 of which ultrasonic wave exit end face is a concave sphere and is coated with SiO2 AR. A cell 19 to be dissected is prepared by growing the same in cell culture solution 20 in a dish 18 and is put on a specimen stage 15. FIG. 2 shows a state in which the boundary of a target cell and other cells is irradiated with the ultrasonic wave converged by the acoustic lens 12.

The controller 3 is means for controlling the ultrasonic wave generation means 1 such that the ultrasonic wave converged by the ultrasonic wave convergence means 2 generates the acoustic streaming having a converged spot diameter effective for relatively removing the target biological object. Since generation of such acoustic streaming is essential in the dissection of the present invention, the controller 3 is important.

The term “acoustic streaming” is intended to mean a thin flow of liquid generated in the liquid by the converged ultrasonic wave. “The acoustic streaming having the converged spot diameter effective for relatively removing the target biological object” is “the acoustic streaming capable of dissecting the biological object”. Such acoustic streaming is the flow of liquid generated by a nonlinear effect of the ultrasonic wave of which converged spot diameter (outer diameter on a cross section taken on a portion at which this acts on the biological object) has a size smaller than a size of an individual biological object (20 μm or less, preferably approximately 5 μm), and the flow having pressure to remove the biological objects from each other.

The controller 3 controls the electrical signal to emit the ultrasonic wave for generating “the acoustic streaming capable of dissecting the biological object”. The electrical signal for emitting the ultrasonic wave for generating the acoustic streaming is the electrical signal having a single frequency maintained for a predetermined period (burst wave). Specifically, the controller 3 controls the electrical signal, thereby controlling a frequency, an emission time, an output (power) and the like of the ultrasonic wave. For example, the controller controls a duration of the ultrasonic wave based on a relationship between a frequency f of the ultrasonic wave generated by the ultrasonic wave generation means 1, NA of the ultrasonic wave convergence means 2 and an underwater ultrasonic wave speed cw, and the output of the ultrasonic wave. In FIG. 5, the arbitrary waveform generator 13, which is the controller, may generate the burst wave having an arbitrary duration by applying the signal shown in FIG. 6 to the RF switch 11a-2. That is, the arbitrary waveform generator 13 serves as a controller of the emission time. Meanwhile, the frequency, the emission time, the output (power) and the like of the ultrasonic wave may be controlled by a computer provided with a control program of the electrical signal and control signal generation means and an electrical signal generator/transmitter capable of being controlled by the computer, in place of the arbitrary waveform generator 13.

The drive unit 4 drives the ultrasonic wave convergence means 2 in XYZ-axis directions. The drive unit 4 drives the ultrasonic wave convergence means 2 in an XY-axis direction (horizontal direction) for irradiating a desired position of the biological objects (boundary of the target biological object and other biological objects) with the ultrasonic wave, and drives the ultrasonic wave convergence means 2 in a Z-axis direction (acoustic wave axis direction) such that the ultrasonic wave is converged at an appropriate height above the biological object. Meanwhile, although the ultrasonic wave convergence means is driven in the XYZ-axis directions in order to converge the ultrasonic wave on a desired position of the biological objects in this embodiment, it is also possible to drive the specimen stage 5 to be described later.

The specimen stage 5 is a flat stage on which the carrier for holding the solution containing the biological objects is placed. It is preferable that the specimen stage 5 has the same configuration as that of a stage of an inverted microscope such that the cell may be observed from below.

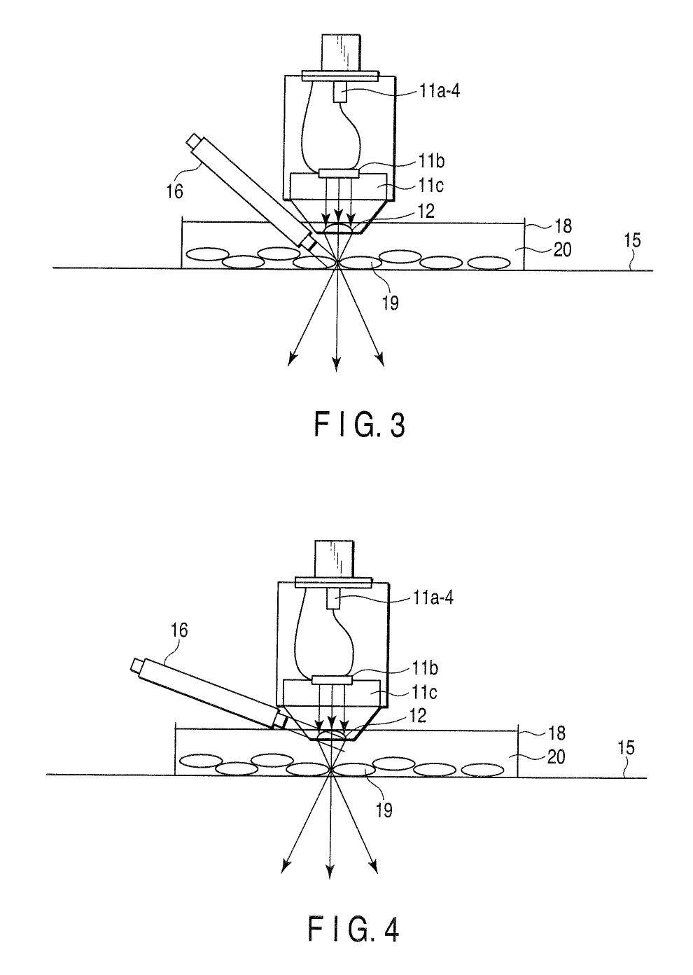

The illumination means 6 illuminates the biological objects in order to clearly observe the biological objects. The illumination means specifically is a light source. The NA of a condenser lens of observation means (microscope) to be described later is smaller than the NA of the ultrasonic wave convergence means (acoustic lens) and it is difficult to illuminate the specimen, so that the dissection device of the present invention is preferably provided with separate illumination means even when this is provided with the observation means (microscope). The illumination means may be composed of one light source for illuminating the biological objects from one direction or may be composed of a plurality of light sources arranged in a circular manner for illuminating the biological objects from various directions. Also, the illumination means may be arranged to directly illuminate the biological objects, as shown in FIG. 3, or may be arranged to illuminate the ultrasonic wave exit end face of the acoustic lens, which is the ultrasonic wave convergence means, to illuminate the biological objects by scattering light thereof, as shown in FIG. 4.

The observation means 7 is means for observing the biological objects, which specifically is an optical microscope objective lens, and preferably is an inverted microscope objective lens. The observation means 7 may be provided with a component of an optical microscope (such as an imaging device) other than the objective lens. The dissection device of the present invention may be provided with the observation means, and when not provided with the observation means, the device may be provided with means for arranging existing observation means in a predetermined position to assemble. When the device of the present invention is not provided with the observation means, it is desirable that the device has a space, jig or the like for arranging at least a part of the observation means (such as the objective lens) on a sound axis of the acoustic lens in an ultrasonic wave radiation direction such that they are assembled with the existing observation means and are used.

2. Ultrasonic Dissection Method

An ultrasonic dissection method of the present invention is a method for generating the acoustic streaming by the converged ultrasonic wave in the solution containing a mass of biological objects spread on the flat carrier, thereby removing the target biological object from a mass of biological objects, including:
(1) a step of converging the ultrasonic wave on the boundary of the target biological object and other biological objects (including not only the boundary but also neighborhood of the boundary (mainly, outside of the target biological object); and
(2) a step of generating the acoustic streaming having the converged spot diameter effective for relatively removing the target biological object in the solution containing the biological objects by the converged ultrasonic wave to relatively remove the target biological object from other biological objects.

The above-described method may be carried out with the ultrasonic wave dissection device of the present invention. Hereinafter, this will be described in the order of steps.

(1) Step of Converging Ultrasonic Wave

The ultrasonic wave (UW) generated by the ultrasonic wave generation means of the ultrasonic wave dissection device is converged on the predetermined focal position (boundary of the target biological object and other biological objects) by the acoustic lens, which is the ultrasonic wave convergence means. In order to converge the ultrasonic wave on the predetermined focal position, the acoustic lens is arranged in a predetermined position. This positioning may be performed by driving the acoustic lens itself in the XYZ-axis directions or by driving the specimen stage in the XYZ-axis directions while observing the biological objects by the observation means.

A positional adjustment of the acoustic lens in the Z-axis direction (acoustic wave axis direction) is important for irradiating a specimen solution with the converged ultrasonic wave to generate the “acoustic streaming capable of dissecting the biological object” in the specimen solution. That is, it is necessary that a distance between the acoustic lens and the biological object to be dissected be controlled to be a focal distance of the acoustic lens. For example, the distance is controlled to be shorter than 5 mm, to be a few millimeters such as 0.5 mm. At that time, a part of the acoustic lens (at least a portion including the ultrasonic wave exit end face) is immersed in the solution containing the biological objects.

The converged spot diameter of the converged ultrasonic wave (outer diameter on the cross section taken on the portion at which this acts on the biological object) has a size effective for relatively removing the target biological object, that is, the size smaller than the size of the individual biological object (20 μm or less, preferably, approximately 5 μm).

(2) Step of Generating Acoustic Streaming

In order to generate the “acoustic streaming capable of dissecting the biological object”, that is, the “acoustic streaming having the converged spot diameter effective for relatively removing the target biological object” from the converged ultrasonic wave, it is necessary to appropriately set the frequency, the emission time, the output (power) and the like of the ultrasonic wave, as described above.

Although the frequency of the ultrasonic wave is appropriately set by one skilled in the art to generate the “acoustic streaming capable of dissecting the biological object”, this is preferably set to be higher than a value “5 MHz” or “10 MHz” described as the frequency to generate the acoustic streaming, in The Technical Report, UE93-93, EA93-93 (1994-01), The Institute of Electronics, Information and Communication Engineers. The frequency is, for example, several hundred MHz, and although 300 MHz is used in an example to be described later, the value is not limited to this.

Although the emission time (hereinafter, also referred to as the duration) of the ultrasonic wave is appropriately set by one skilled in the art to generate the “acoustic streaming capable of dissecting the biological object”, time to generate the acoustic streaming is needed in general, and this is longer than a few μs (such as approximately 0.5 μs) used in the ultrasonic wave microscope, and a period of 10 μs or longer is needed in the present invention. As in the example to be described later, a minimum duration required for the removal of the cell differs depending on the output (power); however, it is found by the inventors of the present invention that the duration is not unlimitedly shortened with increase in the output (power), but is asymptotic to approximately 10 μs. However, when the ultrasonic wave is unnecessarily continuously emitted after generating the acoustic streaming, a width of the acoustic streaming is increased and the acoustic streaming having a narrow width capable of removing the biological object is lost, so that it is not preferable to make the emission time too long. The emission time is, for example, several hundred ps to several hundred ms, and although 400 μs or 300 μs is used in the example to be described later, the value is not limited to this.

Although the output (power) of the ultrasonic wave is appropriately set by one skilled in the art to generate the “acoustic streaming capable of dissecting the biological object”, high power to generate the above-described acoustic streaming, that is, the high power to generate the nonlinear effect of the ultrasonic wave is required in general. In order to generate the above-described acoustic streaming, the high power ultrasonic wave is preferably used in the present invention. Although the power of 0.4 to 1.0 W is used as the power of the electrical signal in the example to be described later, the value is not limited to this. However, technically, not the whole power of 0.4 to 1.0 W is used to generate the acoustic streaming. That is, not the whole electrical signal generated by the ultrasonic wave generation means is transduced to the acoustic wave, and a part thereof is lost. In addition, a part of the ultrasonic wave converged by the acoustic lens is lost due to reflection or the like and not the whole ultrasonic wave is used to generate the acoustic streaming.

In the present invention, it is preferable that the frequency (f) of the ultrasonic wave generation means, the numerical aperture (NA) of the ultrasonic wave convergence means (acoustic lens), and the underwater ultrasonic wave speed cw=1500 m/s satisfy a relational expression, cw/2(fNA)<20 μm* (* is resolution of definition of Sparrow), and that the ultrasonic wave is applied for the duration of 10 μs or longer.

In order to relatively remove the target biological object from other biological objects, if necessary, the boundary of the target biological object and other biological objects is irradiated with the ultrasonic wave a plurality of times (for example, until the removal of the target biological object is confirmed).

After removing the target biological object from a mass of biological objects, the acoustic streaming may be generated in a wide range by the ultrasonic wave radiation with a longer emission time to eliminate other biological objects from the carrier, thereby leaving only the target biological object on the carrier. Effect of eliminating the biological objects other than the target biological object may be improved by increasing the power of the ultrasonic wave or by increasing a radiation time.

### EXAMPLE 1

An example of performing the dissection of the cell with the ultrasonic dissection device shown in FIG. 5 will be described below.

The dissection device shown in FIG. 5 is composed of the signal generator 11a-1 which generates the electrical signal, the arbitrary waveform generator 13 capable of controlling the emission time as described above, the RF switch 11a-2, the power amplifier 11a-3 which amplifies the electrical signal, the ZnO thin-film transducer 11b which transduces the generated electrical signal to the acoustic wave, the sapphire rod 11c which propagates the acoustic wave to the acoustic lens, the acoustic lens 12 which converges the acoustic wave, the drive unit 14 to drive the acoustic lens 12, the specimen stage 15 on which the dish 18 for accommodating the cell culture solution 20 containing the cell 19 is placed, and an objective lens 17 of the inverted microscope to observe the cell.

FIG. 6 shows a state in which the burst wave is formed from the electrical signal generated by the signal generator, in the dissection device shown in FIG. 5. That is, FIG. 6 shows that the electrical signal having the single frequency generated by the signal generator is controlled to be output only during a predetermined duration by the arbitrary waveform generator to form the burst wave.

Hereinafter, an experimental procedure will be described in detail.

The objective lens 17 of the inverted microscope was raised from a cell position by work distance (WD). With this operation, the acoustic wave is focused on the cell position. Next, the objective lens was focused on an innermost part of the acoustic lens to adjust a horizontal position such that a focal position was on a central portion of an observation plane.

The objective lens was refocused on the cell, shot as a test with a slightly less than 1 W through the signal having the frequency of 300 MHz and the duration of 400 μs, and the position was further adjusted. Specifically, the signal with the frequency of 300 MHz was emitted from the signal generator 11a-1, and the signal according to an input format of the RF switch 11a-2 was given by the arbitrary waveform generator 13, thereby making the signal of 300 MHz a burst wave with a predetermined duration (=emission time). The burst wave was amplified by the power amplifier 11a-3 and was transduced to an acoustic plane wave by the transducer 11b. The plane wave passed through the acoustic lens 12 and became a converged acoustic wave in the cell culture solution 20, and was converged in the vicinity of the cell 19. Herein, when the frequency, the duration and the power are appropriate, it is possible to form thin acoustic streaming acting on one cell just below the acoustic lens. In this example, the frequency of 300 MHz, the duration of 400 μs, and incident power of 0.4 to 1.0 W were used. The power is the power of the electrical signal, and loss in the transducer and the loss in the acoustic lens are not taken into account.

After finishing the adjustment, the target cell (group) was selected, and the target cell was removed from adjacent cells by irradiating the boundary of the target cell and the adjacent cells with the ultrasonic wave a plurality of times for each cell. As for the cells in a range distant from the target cell, the cell group in a wide range was eliminated at once by increasing the power, by defocusing the acoustic lens in a direction to be separated from the cell, or by increasing the duration, thereby eliminating the cells other than the target cell. Since the eliminated cells floated on the cell culture solution, they were eliminated by liquid exchange. By the above-described procedure, only the target cell was successfully left on the carrier.

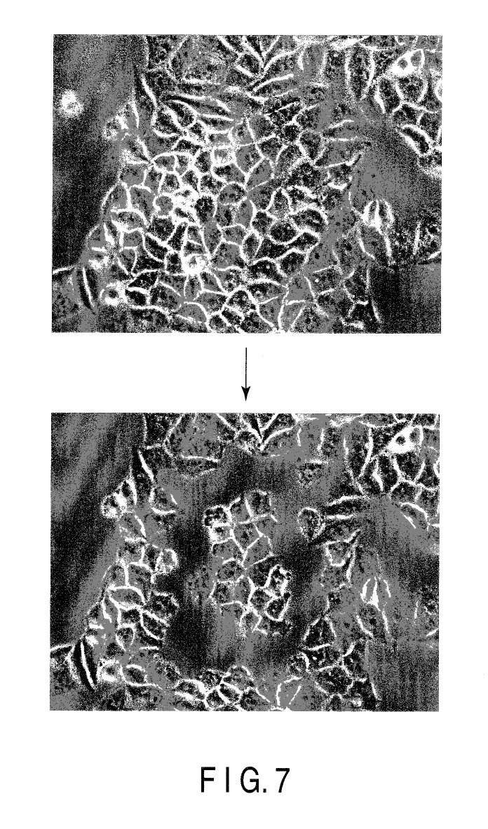

The experimental result is shown in FIG. 7. An upper photograph in FIG. 7 shows the cells before the dissection, and a lower photograph shows the cells just after the dissection. FIG. 7 shows that the target cell is removed from the adjacent cells. Meanwhile, although a plurality of target cells are removed from other cells in this embodiment, as described in Example 2 to be described later, it is also possible to remove the single target cell from other cells according to the method of the present invention.

### EXAMPLE 2

In this example, an experiment was performed to confirm that the damage to the cells was small when removing one target cell from other cells with this method.

With the same experimental arrangement as that of the above-described Example 1, the cell dissection was performed following the procedure similar to that of Example 1. An experimental parameter is set to 300 MHz, 2.5 W, and the duration of 300 μs. After the dissection, the cells were conserved in the container until the following day.

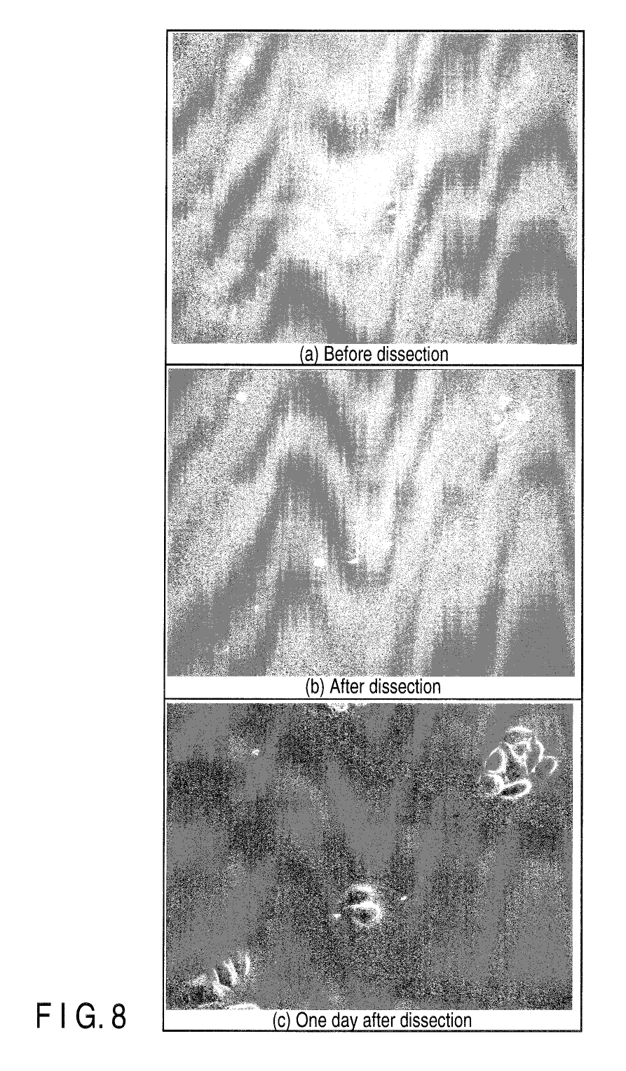

The result is shown in FIG. 8. FIG. 8 shows photographs taken before the ultrasonic dissection, just after the same, and one day after the same from top to bottom. It was observed that the cell was divided once one day after the dissection, and it was verified that the damage due to the dissection was small.

### EXAMPLE 3

In this example, the minimum duration of the ultrasonic wave required for removing the target cell from other cells was checked by varying the output of the electrical signal. This example also was performed with the same experimental arrangement as that of the above-described Example 1 and following the procedure similar to that of Example 1.

The duration is an important parameter for allowing the acoustic streaming to sufficiently develop. It is considered that with the duration of a certain time or shorter, the acoustic streaming is not sufficiently developed and does not reach the speed with which the cell may be removed. In order to check the minimum duration, the minimum duration with which the cell may be removed when varying the input power was checked.

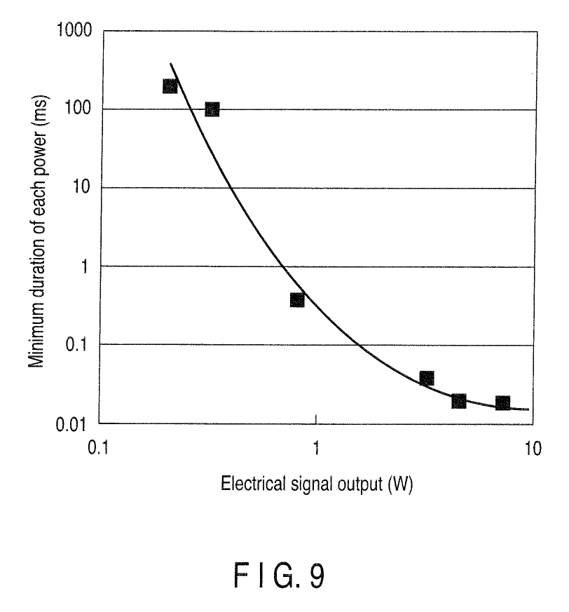

The result is shown in FIG. 9. The graph was asymptotic to 10 ps and it was confirmed that 10 μs is the minimum duration.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An ultrasonic dissection method for generating acoustic streaming by a converged ultrasonic wave in a solution containing a mass of biological objects spread on a flat carrier, thereby relatively removing a target biological object from other biological objects, the method including:
a step of converging an ultrasonic wave on a boundary of the target biological object and other biological objects; and
a step of generating the acoustic streaming having a converged spot diameter effective for relatively removing the target biological object, in the solution containing the mass of biological objects by the converged ultrasonic wave to relatively remove the target biological object from other biological objects.

2. The ultrasonic dissection method according to claim 1, further including a step of, after removing the target biological object from other biological objects, eliminating other biological objects from the carrier to leave only the target biological object on the carrier.
